(19) United States
(12) Patent Application Publication
(10) Pub. No.: US 2016/0041181 A1
(43) Pub. Date: Feb. 11, 2016

(54) NMR ASSAY TO SCREEN PROTEIN-PROTEIN INTERACTION INHIBITORS

(71) Applicant: RUTGERS, THE STATE UNIVERSITY OF NEW JERSEY, New Brunswick,, NJ (US)

(72) Inventors: Gaetano T. Montelione, Highland Park, NJ (US); Patrick L. Nosker, Stockton, NJ (US); Vikas Nanda, Highland Park, NJ (US); James M. Aramini, Glenside, PA (US); Douglas Pike, Somerset, NJ (US); Lichung Ma, South Princeton Junction, NJ (US); Keith Hamilton, Brick, NJ (US); Srinivas Annavarapu, Somerset, NJ (US)

(73) Assignee: RUTGERS, THE STATE UNIVERSITY OF NEW JERSEY, New Brunswick, NJ (US)

(21) Appl. No.: 14/765,626

(22) PCT Filed: Feb. 4, 2014
(86) PCT No.: PCT/US14/14739

§ 371 Date: Aug. 4, 2015

Related U.S. Application Data
(60) Provisional application No. 61/760,204, filed on Feb. 4, 2013.

(51) Int. Cl. G01N 33/68; A61K 38/08

(52) U.S. Cl. CPC G01N 33/6845; A61K 38/08; G01N 2500/02; G01N 2500/20

(57) ABSTRACT

This invention relates generally to a field of rational drug design. In particular, the present invention relates to a ¹⁹ F NMR assay for screening inhibitors of specific protein-target interactions, preferably those that inhibit influenza A NS 1 protein and can be useful in treating influenza A viral infections. The invention also relates to agents, compositions, and methods for treating influenza A viral infections.

### CROSS-REFERENCE TO A RELATED APPLICATION

This application claims the benefit under 35 U.S.C. 119(e) of U.S. Provisional Application No. 61/760,204 filed on Feb. 4, 2013, the content of which is incorporated herein in its entirety.

### STATEMENT OF GOVERNMENT SUPPORT

The invention disclosed herein was made, at least in part, with Government support under Grant Nos. 5R01 GM089949, U54 GM094597 and U01 A1074497 from the National Institutes of Health. Accordingly, the U.S. Government has certain rights in this invention.

### FIELD OF THE INVENTION

This invention relates generally to a field of rational drug design. In particular, the present invention relates to a ¹⁹F NMR assay for screening inhibitors of general protein-target interactions including, but not limited to, Protein-Protein, Protein-Nucleic Acid, Protein-Peptide, and Protein-Small-molecule interactions, with particular application to the inhibition of influenza A NS1 proteins.

### BACKGROUND

Protein-protein (PPI), protein-nucleic acid (PNI), and Protein-Small Signaling Molecule (PSI) interactions play critical roles in macromolecular recognition throughout nature. Although the number of well-characterized examples is still relatively modest, it is becoming apparent that many different kinds of interactions can be inhibited using drug-like small molecules. However, such interactions have traditionally been shunned by many small-molecule drug developers, despite their therapeutic relevance and untapped abundance, largely because of technological hurdles. Compared to active site targeting, PPI and PNI inhibition, for example, suffers from the particular problem of more exposed and less defined binding sites, and this imposes significant experimental challenges to the development of the interaction inhibitors.

In the specific case of influenza (commonly known as “the flu”), which is an infectious disease of birds and mammals caused by segmented negative-stranded RNA viruses of the Orthomyxoviridae family, the viral genome is comprised of up to 14 proteins. These proteins are either incorporated into the virion particle or expressed in the infected host cell, so-called “structural” and “non-structural” proteins, respectively. One of such proteins is a multifunctional non-structural protein 1 of influenza A virus (NS1A).

NS1A plays an integral role in subverting the innate antiviral response of the host and also in regulating several virus functions, including sequestering the Cleavage and Polyadenylation Specificity Factor 30 (CPSF30) protein. Another mechanism of suppressing innate immune response to viral infection involves cooperative binding to dsRNA molecules, which are known to otherwise stimulate the innate immune response in human cells. NS1A is made up of two key domains, a RNA-binding domain (RBD) and an effector domain (ED). The influenza virus NS1A ED inhibits interferon production by binding cellular CPSF30 protein, which is necessary for maturation of interferon RNAs (see U.S. Pat. Nos. 7,709,190, 7,601,490, and 8,455,621; each incorporated by reference in its entirety). The NS1A protein specifically binds a region of CPSF30 that includes an alpha-helix in the F3 zinc-finger domain.(Das, K. et al. Proc. Natl. Acad. Sci. U.S.A. 2008, 105: 13092-13097; incorporated by reference in its entirety and U.S. Pat. Nos. 7,709,190, and 8,455,621) The NS1A ED includes residue tryptophan (Trp) 187 in its CPSF30-binding site (Das, 2008). The RBD and ED together work to disable host cell defenses. By targeting the NS1A protein, influenza infection progression can be stopped. These studies coupled with recent progress in the design of NS1A-based inhibitors (Jablonski, J. J., et al. (2012). Bioorg Med Chem 20, 487-497; incorporated herein by reference in its entirety) and attenuated viruses (Richt, J. A., et al. (2009) Curr Top Microbiol Immunol 333, 177-195; incorporated herein by reference in its entirety), illustrate the importance of the NS1A protein as a target for the development of novel therapeutics to combat future outbreaks of potentially deadly forms of influenza A virus.

There is, therefore, a continuing need for an effective and simple method to identify compounds (preferably drug-like small molecules) that can in interfere in Protein-protein/peptide (PPI), protein-nucleic acid (PNI), and Protein-Small Signaling Molecule (PSI) interactions that play critical roles in macromolecular recognition. In particular, there is a continuing need for an effective and simple method to identify compounds that can interact with NS1A protein and its various PPIs and PNIs, and thereby can be used in development of therapeutics to suppress influenza infection.

### SUMMARY

In view of the above-described problems, needs, and goals the inventors have devised embodiments of the present invention in which inhibitors of protein interactions (protein-protein, protein-nucleic acid, and protein small molecule) can be effectively identified based on a novel assay that relies on labeling a protein with a fluorine isotope (¹⁹F) and monitoring Nuclear Magnetic Resonance (NMR) signal (e.g., chemical shift, line shape, and signal relaxation) of the protein in the presence of one or more candidate compounds (i.e., potential inhibitors). In another embodiment of the present invention, the inhibitors of protein interactions can be effectively identified based on labeling a target of the protein with a fluorine isotope (¹⁹F) and monitoring NMR signal of the target in the presence of one or more candidate compounds. In yet another embodiment of the present invention, the inhibitors of protein interactions can be effectively identified based on labeling one or both the protein and the target of the protein with a fluorine isotope (¹⁹F) and monitoring their NMR signal in the presence of one or more candidate compounds. In one exemplary embodiment, the novel assay is applied to the interactions between the influenza A virus NS1 protein and either itself or with host proteins

The ¹⁹F isotope is a valuable NMR probe for biological systems due to its numerous favorable properties, including its nuclear spin (I=½), high natural abundance (100%), extremely high resonance frequency and sensitivity (83% that of ¹H), minimal inherent ¹⁹F background signals, and the exquisite sensitivity of its chemical shift to changes in local environment. In particular, 1-dimensional (1D) ¹⁹F NMR spectra are simple and high sensitivity, making the suitable for screening compound libraries for potential inhibitors of PPIs, PNIs, and PSIs. Moreover, because of the comparable atomic radii of hydrogen and fluorine, incorporation of fluorinated amino acids into proteins generally results in relatively minor structural perturbations.

The method generally includes three following steps:

(i) labeling a protein and/or its target (e.g. protein, nucleic acid, peptide, and signaling molecule) at one or more sites with the fluorine isotope (i.e., exchanging one or more natural amino acids with fluorine substituted amino acids);

(ii) providing a reaction system comprising (a) the protein, (b) one or more candidate compounds (i.e., potential binding inhibitors), and (c) the protein binding target (e.g. protein, nucleic acid, peptide, and/or signaling molecule), where at least protein, at least target or both the protein and the target are labeled with the fluorine isotope; and

(iii) monitoring interaction of the protein with its binding target using the changes in the ¹⁹F NMR signal (e.g., chemical shift perturbation, line shape, and signal relaxation). A reduced binding level in the presence of the candidate compound relative to a control binding level is indicative of the inhibitory activity of the compound in suppressing the protein complex formation.

In one exemplary embodiment, the method includes (i) labeling Non-Structural protein 1A (NS1A) of the influenza A virus at one or more sites with the fluorine isotope; (ii) providing a reaction system comprising (a) the ¹⁹F-labeled NS1A, (b) one or more candidate compounds, and the NS1A binding partner, preferably a cleavage and polyadenylation specificity factor 30 (CPSF30) or a fragment thereof; and (iii) monitoring interaction of the NS1A protein with its binding partner. For example, in the case of the NS1:CPSP30 interaction the reduced binding level in the presence of the candidate compound relative to a control binding level is indicative of the inhibitory activity of the compound against influenza A virus.

In the alternative exemplary embodiment, instead of labeling NS1A, its binding partner (e.g. CPSF30 or a fragment thereof) is labeled with the ¹⁹F fluorine isotope. In yet another alternative exemplary embodiment, both NS1A and its binding partner (e.g. CPSF30 or a fragment thereof) are labeled with the same or different fluorinated amino acids.

In such embodiments, the hydrogen atom is replaced with a fluorine isotope at one or more amino acids either at their backbone atoms, side-chain atoms or both. Preferably, the hydrogen atom is replaced with a fluorine isotope at the side chains of such amino acids due to their exposure in the binding interface. In some embodiments, one type of the amino acid residues is labeled on the protein of interest and another type of the amino acid on the binding target. In a preferred embodiment, due to the general importance of aromatic residues at protein interfaces combined with the relative paucity of the aromatic amino acids in proteins, the hydrogen atom is replaced with a fluorine isotope at the aromatic residues. ¹⁹F NMR and incorporation of fluorinated aromatic amino acid analogs provides high-sensitivity, simple-to-detect probes of protein interaction surfaces in complexes, including dimers. In one exemplary embodiment, the amino acids are selected from tryptophan and phenylalanine, and more preferably 5-fluoro-tryptophan (5FW) and/or 4-fluoro-phenylalanine (4FF). In another exemplary embodiments, one type of the aromatic residues (e.g., W) is labeled on the protein of interest and another type of the aromatic residues (e.g., F) is labeled on its binding partner. In one preferred embodiment, the aromatic residues at the CPSF30 binding pocket of the NS1A protein are labeled with the fluorine isotope. The labeled residues on the NS1A protein are selected from W102, W187, W203 or a combination thereof. In another preferred embodiment, phenylalanine residues in or near the NS1-binding site of CPSF30 or a fragment of CPSF30 are labeled with the fluorine isotope.

It is also within the scope of this invention that instead of using the whole protein, the reaction system may comprise a plurality of polypeptides having the binding portions, where at least one of the polypeptides is labeled with ¹⁹F and a candidate compound. In case of the NS1A protein, the reaction system may comprise a plurality of polypeptides having NS1A-dimerization portions, where at least one of the polypeptides is labeled with 5-fluoro-tryptophan (5FW) and/or 4-fluoro-phenylalanine (4FF), and a candidate compound. Thereby, the method relies on detecting dimerization of these polypeptides. A reduced dimerization level in the presence of the candidate compound relative to a control dimerization level is indicative of activity of the compound against influenza A virus.

The disclosed invention is also directed to candidate compounds identified using the disclosed NMR assay. The invention also provides a pharmaceutical composition comprising (i) such candidate compounds and (ii) a pharmaceutically acceptable carrier.

The invention further provides a method of preventing or treating influenza A infection. The method includes identifying a patient in need of such prevention or treatment, and administering to the patient a first therapeutic agent comprising a therapeutically effective amount of the isolated peptide or the pharmaceutical composition described above.

The preferred methods and materials are described below in examples which are meant to illustrate, not limit, the invention Skilled artisans will recognize methods and materials that are similar or equivalent to those described herein, and that can be used in the practice or testing of the present invention. Unless otherwise defined, all technical and scientific terms used herein have the same meaning as commonly understood by one of ordinary skill in the art to which this invention belongs. Other features and advantages of the invention will be apparent from the detailed description and from the claims.

### BRIEF DESCRIPTION OF THE DRAWINGS

FIG. 1A shows an amino acid sequence of NS1A protein from 4 different representative strains of influenza A virus.

FIG. 1B shows an amino acid sequence of NS1A protein from UDORN/307/1972 strain with tryptophan residues identified.

FIG. 2 shows a protocol for aromatic ¹⁹F labeling of NS1A protein using E. coli BL21(DE3)-Gold expression system.

FIG. 3A is a sequence of 5 1D spectra of the concentration dependence of the ¹⁹F NMR signal of Trp187 within 5FW-labeled Ud NS1A ED. These data demonstrate the use of ¹⁹F NMR to monitor a protein dimerization or self-association process. Resonances corresponding to Trp187 in the dimer and monomer states are denoted by ‘D’ and ‘M’, respectively.

FIG. 3B is a comparison of ¹⁹F NMR spectra of 5FW-labeled monomeric 25 μM Ud NS1A ED (top) and 500 μM [K110A] NS1A ED (bottom) in high salt pH 8 buffer; the K110A mutant is known to disrupt the homodimer of the NS1A ED, and this disruption can be monitored by ¹⁹F NMR.

FIG. 3C is a fit plot of the fraction of ¹⁹F dimer resonance volume as a function of total Ud NS1A ED concentration, demonstrating the use of ¹⁹F NMR to monitor dimer association. Dashed lines represent 95% confidence bounds for the fit.

FIG. 4A shows a diagram of influenza A NS1 targeting of CPSF30 to suppress processing of mRNA of antiviral response proteins, including beta-interferon.

FIG. 4B shows a diagram of the CPSF complex on pre-mRNA in a pre-mRNA processing.

FIG. 5 shows the elution profile of NS1A alone and in complex with F2F3 fragment of CPSF30.

FIGS. 6A-6B show the structure of the tetrameric complex formed between NS1A effector domain (ED) and the F2F3 fragment of CPSF30.

FIG. 6C shows the structure of the NS1A RBD and ED domains with Trp residues highlighted.

FIGS. 7A-7D show the effects of amino acid substitutions in NS1A on its interaction with CPSF30 and on its function in Influenza A virus-infected cells. (A) GST-F2F3 pulldown assay showing binding between F2F3 fragment of CPSF30 and NS1A for with wild-type (wt) NS1A protein only, and not for mutants of NS1A in the F2F3-binding site of NS1A; (B) plaques of the wild-type and G184R mutant Ud viruses in MDCK cells demonstrating that mutation in the F2F3 binding sites of NS1 results in attenuated influenza A virus; (C) an SDS gel showing a control experiments verifying that G184R mutation in the Ud NS1A protein does not affect the amount of the NS1A protein synthesized in MDCK cells infected with 5 pfu/cell; and (D) quantitative RT-PCR measuring amounts of IFN-beta pre-mRNA (left) and IFN-beta mRNA (right) in wild-type and G184R Ud-infected cells, demonstrating that flu virus with wt NS1A block the processing of IFN-beta mRNA, but flu virus with a G184R mutation in the CPSF30 binding site of NS1A no longer block process of INF-beta pre mRNA to mature mRNA.

FIG. 8 is an expanded region from the structure of FIG. 6 illustrating details of the interactions between the ED of NS1A and the F2F3 fragment of CPSF30, and the location of residue W187 of NS1A in this CPSF30-binding site.

FIG. 9 show the structure of the tetrameric complex formed between NS1A effector domain (ED) and the F2F3 fragment of CPSF30 with positions of 5FW (dark grey) and 4FF (light grey) identified.

FIG. 10 show stack plot of ¹⁹F NMR spectra of 5FW-labeled NS1A ED (top), 4FF-labeled F2F3 (bottom), and a 4FF-F2F3:5FW-ED complex (center). Changes in ¹⁹F chemical shifts of both 5FW and 4FF occur upon complex formation.

FIG. 11 show stack plot of ¹⁹F NMR spectra of full-length NS1A (top), full-length W16A mutant NS1A (center) and ED domain only of NS1A (bottom). The data shows no ED:ED dimeric interaction in the full length protein at dilute (25 μM) condition.

FIG. 12A show a diagram of cooperative binding of NS1A to dsRNA. The location of Trp187 of NS1A, which when labeled with ¹⁹F provides the basis for an assay of NS1A:F2F3 interactions or NS1A:NS1A self association, is indicated

FIG. 12B is an Electrophoretic gel mobility assays (EMSA) showing that Trp187 in NS1A, and consequently intermolecular ED:ED interactions, is required for cooperative dsRNA binding by the full-length protein. Top left—wild type Ud NS1A(1-215). Top right—, [W187R]Ud NS1A(1-215). Bottom—Ud NS1A(1-73) dsRNA-binding cooperativity and binding affinity is reduced either by mutating residue Trp187 (top panels) or by deletion of the ED from the NS1A molecule (bottom panel)

FIG. 13 shows the F2F3 binding site on NS1A and a portion of the bound structure of F2F3, including a key helical region (from F3 fragment of CPSF30) involved in the interaction. The location of 3 Phe residues of F2F3 in the NS1A:F2F3 interface, which when labeled with ¹⁹F provides the basis for an assay of NS1A:F2F3 interactions, is indicated.

FIG. 14 shows a comparison of the natural CPSF30 ligand fragment from FIG. 13 with two candidate peptides designed using a D-amino acid N-terminal cap as well as a D-amino acid C-terminal cap with three key residues shown to provide significant binding activity in the structure of the NS1A-F2F3 complex.

FIG. 15A shows ¹⁵N—¹H HSQC NMR spectrum of the Effector Domain (ED) of NS1A (full spectrum at top, and expanded region at bottom) in the presence and absence of various amounts of (i) a test peptide or (ii) a control peptide, demonstrating chemical shift perturbations of backbone ¹⁵N and ¹H resonances of amino acid residues located near the F2F3 binding site/homodimerization site, resulting from the peptide binding to the F2F3-binding site of NS1A.

FIG. 15B shows chemical shift perturbation chart created using ¹⁵N—¹H HSQC shift data obtained for NS1A ED recorded with and without addition of the test peptide. These data demonstrate that the Peptide binds to the NS1A ED in the same region of ED that is known to be involved in interactions with F2F3 and/or in the homodimerization of the NS1A ED.

FIG. 16A shows an overlay of 1D ¹⁹F-Trp NMR spectra of the ED of NS1A ED labeled with 5FW, with increasing concentrations of the F2F3 fragment of CPSF30. The ¹⁹F resonance assignments of residues W102, W187, and W203 were determined by single residue mutagenesis and are labeled in these spectra. The data demonstrate a means of assaying formation of the tetrameric complex between the ED and F2F3 by changes in the chemical shift values of all three of the ¹⁹F-Trp resonances.

FIG. 16B shows overlays of ¹⁹F NMR spectra as in FIG. 14A in presence of inhibitor peptide. These data demonstrate the use of ¹⁹F NMR spectra to monitor binding of a designed peptide in or near residue Trp187, which is in both the CPSF30 and self-dimerization binding sites of NS1A.

FIGS. 17A-17B show the benefit of NONA peptide therapeutics (A), and using D-amino acids as caps can increase hydrogen bonding and helix stability (B).

### DETAILED DESCRIPTION OF THE INVENTION

A novel assay centered on ¹⁹F labeling of (a) biologically important protein(s) is disclosed that can effectively identify a compound, an agent, or a composition for treating a disease state by interfering with a specific protein-protein or protein-nucleic acid. An example disease/drug development system is the design of therapeutics targeting a specific pathogen (e.g., influenza A virus) based on changes in protein-protein interactions between a viral protein (e.g. influenza NS1A) and its host cell target (i.e., binding partner), such as human CPSF30 protein. The assay is demonstrated on a targeted rational drug in silico design for peptide-like inhibitors that interfere with the interaction between a pathogenic protein (e.g. influenza NS1A) and its target in host cells. Generally, the assay relies on changes in molecular dynamics and biomolecular NMR in the presence of one or more candidate compounds (i.e., potential inhibitors). Specifically, the assay relies on labeling a protein, such as NS1A of an influenza A virus, and/or its binding partner(s) with a fluorine isotope (¹⁹F), preferably at the aromatic residue at or near the binding pocket, and monitoring NMR signal changes in the presence of one or more candidate compounds (i.e., potential inhibitors). The assay may be done either with the protein alone, its binding partner alone, or using a complex of the protein with it binding partner and assaying for disruption of the complex.

The method generally includes (i) labeling protein, such as non-structural protein 1A (NS1A) of an influenza A virus, at one or more sites with the fluorine isotope (i.e., exchanging one or more natural amino acids with fluorine substituted amino acids); (ii) providing a reaction system comprising (a) labeled protein, (b) one or more candidate compounds (i.e., potential binding inhibitors), and a target binding partner, preferably a cleavage and polyadenylation specificity factor 30 (CPSF30); and (iii) monitoring protein:target interaction of the protein with its binding target. A reduced binding level in the presence of the candidate compound relative to a control binding level is indicative of the inhibitory activity of the compound against the protein and if the protein is a pathogenic protein, against the pathogen. In the alternative embodiment, instead of labeling the protein, its binding target can be labeled with the fluorine isotope. In yet another alternative embodiment, both protein and its binding partner are labeled. Each step of the disclosed method will now be described in more detail.

(i) Labeling Protein and/or Its Target

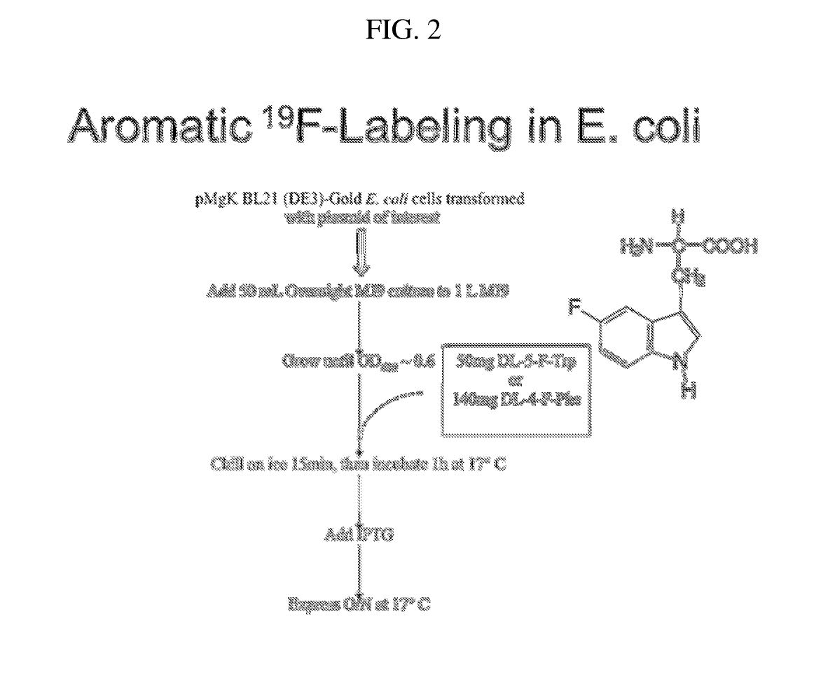

At this stage of the disclosed assay, the labeled protein and/or its target can be prepared by a variety of techniques known in the art. Examples include protein expressions in Escherichia coli cells containing a plasmid encoded with said protein. (see Example 1). During culturing, the cells are provided an alternative source of amino acids such as 5-fluoro-DL-tryptophan, followed by incubation and induction of protein expression (see FIG. 2). While the disclosed method of synthesizing the labeled protein is described with reference to a bacterial expression system, it is to be understood that the protein can also be prepared using alternative expression systems (e.g. pichia or insect cells) or using a peptide synthesizer based on solid phase synthesis.

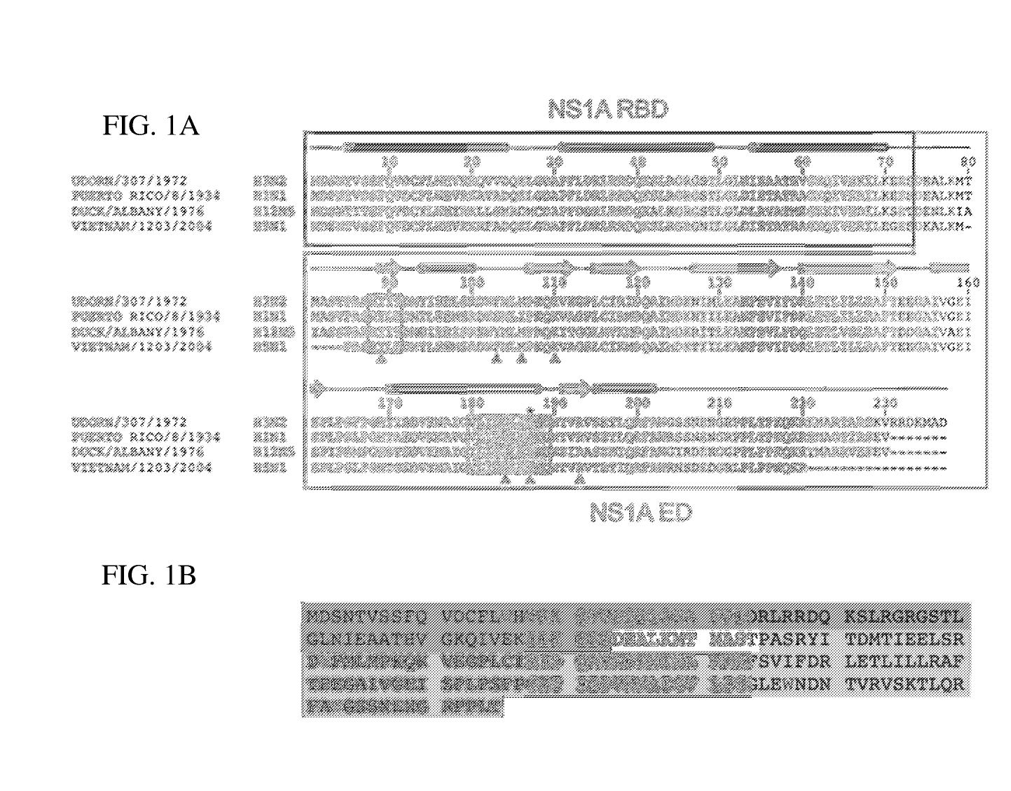

In one exemplary embodiment, the protein is non-structural protein 1A (NS1A) of an influenza A virus. Examples of influenza A strains that can be used with the discloses assay include, but not limited to, A/Memphis/8/88, A/Chile/1/83, A/Kiev/59/79, AAUdorn/307/72, A/NT/60/68, A/Korea/426/68, A/Great Lakes/0389/65, A/Ann Arbor/6/60, A/Leningrad/13/57, A/Singapore/1/57, A/PR/8/34, A/Vietnam/1203/04, A/HK/483/97, A/South Carolina/1/181918 (the 1918 pandemic virus H1N1 strain), and A/WSN/33. In particular, influenza A strains include mammalian Influenza A virus, e.g., H3N2, H1N1, H2N2, H7N7 and H5N1 (avian influenza virus) strains and variants thereof. The sequences of these strains are available from GenBank, CDC and viral stock may be available from the American Type Culture Collection, Rockville, Md. or are otherwise publicly available. For example, the amino acid sequence of the NS1A protein of Influenza A virus, A/Udorn/72 is provided in FIG. 1A.

The NS1A protein has four tryptophan residues and seven phenylalanine residues. In one embodiment, one or more of these residues, and specifically their side chain aromatic rings, are labeled with the isotope ¹⁹F. Preferably, the residues at or near the CPSF30 binding epitope (“CPSF binding site”), which is a portion of the NS1A protein that interacts with one or more zinc fingers of the CPSF30 protein, are labeled. Alternatively, or in combination, the residues at or near the double-strand RNA binding epitope are labeled. Greater structural details regarding the double-strand RNA binding epitope is provided in U.S. Pat. Nos. 7,709,190 and 7,601,490 and the PCT App. No.: PCT/US2003/036,292, which are incorporated herein by reference. In a preferred embodiment, tryptophan W102, W187, and W203 are labeled with 5-fluoro-DL-tryptophan (see formula below).

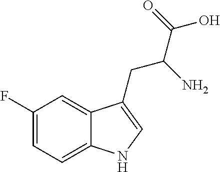 [SMILES: NC(Cc1c[nH]c2ccc(F)cc12)C(=O)O]

The target is not particularly limited as long as it interacts/binds with the protein. In one particular embodiment, the target is a host target and the host is human. In one exemplary embodiment, the target is a cleavage and polyadenylation specificity factor 30 (CPSF30) and the pathogenic protein is NS1A. The amino acid sequence of the CPSF30 is available from GenBank under UniProt Id 095639. In one embodiment, one or more of the aromatic residues in CPSF30, and specifically their side chain aromatic rings, are labeled with the isotope ¹⁹F. Preferably, the residues at or near the NS1A binding epitope (“NS1A binding site”), which is a portion of the CPSF30 protein that interacts with NS1A protein, are labeled.

The CPSF30 protein has a plurality of zinc finger domains that interact with NS1A, namely, “F1”, “F2” and “F3”. These zinc finger domains can interact with NS1A individually or as pairs “F1F2”, “F2F3”, or as a trimer “F1F2F3”. The amino acid positions sequence of these domains include: F1 Zn-Finger Domain: residues 41-59; F2 Zn-Finger Domain: residues 68-86; F3 Zn-Finger Domain: residues 96-114; F4 Zn-Finger Domain: residues 124-142; F5 Zn-Finger Domain: residues 148-166; and F5 Zn-Finger Domain: residues 243-260, as well as F2F3: residues 61-121 and F1F2F3: residues 39-121. Ideal aromatic amino acid candidates for fluorination include: Phe-84, -98, -102, and -112, Trp-71, and Tyr-88, 97, and 99.

### (ii) Providing a Reaction System

At this stage of the disclosed assay, either labeled protein, its target, both or fragments thereof are combined in a solution with one or more candidate compounds. The candidate compound is a molecule that, for example, may inhibit influenza viral growth and the symptoms of influenza viral infections. The candidate compound may be a protein or fragment thereof, a designed synthetic peptide that does not occur in nature (e.g. a peptide containing D amino acid residues or a stapled peptide), a small molecule, or even a nucleic acid molecule. Various commercial sources of small molecule libraries meet the basic criteria for useful drugs in an effort to “brute force” the identification of useful candidate compounds. Screening of such libraries, including libraries generated combinatorially (e.g., peptide libraries, aptamer libraries, small molecule libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds. Candidate compounds may be screened from large libraries of synthetic or natural compounds. One example of a candidate compound library is an FDA-approved library of compounds that can be used by humans. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.) and a rare chemical library is available from Aldrich (Milwaukee, Wis.). Combinatorial libraries are available or can be prepared. Alternatively, libraries of natural candidate compounds in the form of bacterial, fungal, plant and animal extracts are also available from, for example, Pan Laboratories (Bothell, Wash.) or MycoSearch (N.C.), or can be readily prepared by methods well known in the art. Candidate compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

In one embodiment, the reaction system contains one candidate compound. In another embodiment, in contrast to methodologies of prior art, the reaction system can contain two or more candidate compounds, preferably between 2 and 100 compounds, because the assay does not rely on labeling of candidate compounds and can provide high efficiency screening by testing multiple candidate compounds in parallel. Once a batch with an inhibitor identified, each compound can be tested further to assess its inhibitory activity.

In a preferred embodiment, the candidate compound is a peptide. Peptides are naturally found throughout the body in signaling pathways and hormonal control systems. Some examples of peptide therapeutics include those that trigger prolactin release and insulin response. Although they have been used as drugs to treat various diseases, including diabetes mellitus, peptides undergo proteolytic degradation and therefore do not possess long-term stability in the body Like other proteins, they also have structural stability issues, which can prevent peptides from folding properly if they are shorter than 20 amino acids.

In a preferred embodiment, to identify an inhibitor of NS1A protein, the reaction system includes (i) at least a CPSF30-binding portion of the NS1A protein of an influenza A virus, (ii) at least a NS1A-binding portion of human CPSF30, and (iii) a candidate compound, where either the CPSF30-binding portion of NS1A and/or the NS1A-binding portion of CPSF30 are labeled with 5-fluoro-tryptophan (5FW) and/or 4-fluoro-phenylalanine (4FF). The ¹⁹F probe may also be introduced by biosynthetic incorporation of other ¹⁹F-labeled amino acids.

Alternatively, the reaction system includes a plurality of polypeptides having NS1A-homodimerization portions, where at least one of the polypeptide is labeled with 5-fluoro-tryptophan (5FW) and/or 4-fluoro-phenylalanine (4FF), and a candidate compound. Using this approach, a skilled artisan can then detect homodimerization of the polypeptides. A reduced dimerization level in the presence of the candidate drug relative to a control dimerization level is indicative of activity of the compound against influenza A virus.

(iii) Monitoring Protein-Target Interaction

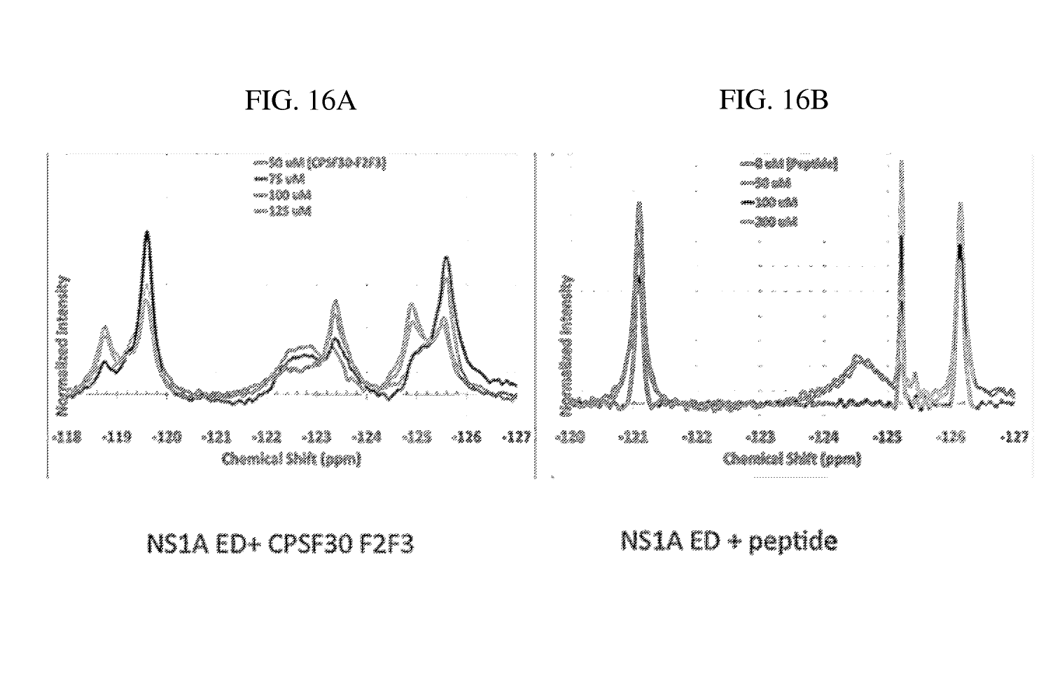

At this stage of the disclosed assay, a combination of the protein, its target, and one or more candidate compounds are exposed to Nuclear Magnetic Resonance (NMR). Within the magnetic field, the nuclei having nonzero spin (i.e., ½) align and can absorb and re-emit a specific resonance frequency. The disclosed assay can be performed in one frequency axis (1D) or multi-frequency axes (2D, 3D) using either direct or indirect detection of the ¹⁹F isotope. Preferably, if the peaks of the labeled residues are sufficiently dispersed on the spectrum (ppm) (see e.g., FIG. 16), then direct detection of ¹⁹F in 1D experiment is desired for its speed, efficiency, and the necessity to only label very view key residues. In such embodiment, if the candidate compound forms a complex with either the protein or its target at their respective epitopes, the changes in the chemical shift of the ¹⁹F atoms indicates binding and potentially inhibition of protein function.

However, it is also within the scope of the present invention to conduct an assay using multi-dimensional NMR experiments, such as heteronuclear single quantum coherence (HSQC) detection using, for example, adiabatic (or composite) 180 degrees pulses, where ¹⁹F occupies one dimension and ¹³C occupies the second dimension. Although, typically, the carbon atoms need to be replaced with ¹³C isotope in such proteins due to ¹²C isotope spin quantum number of zero, the experiments can still be performed using the 1.1% natural abundance of ¹³C isotope. Alternatively, the magnetization can be transferred from the ¹⁹F isotope to either side-chain or backbone nitrogen atom labeled with ¹⁵N. Since larger complexes of pathogenic protein and its host target may have longer rotational correlation times, the assay can also be conducted using transverse relaxation-optimized spectroscopy (TROSY).

Another approach using NMR involves studying nuclear relaxation times. If the candidate compound is able to disrupt the natural complex, and there is binding with reasonable affinity NMR-active nuclei in the system (e.g. ¹⁹F) will exhibit faster nuclear relaxation rates due to longer rotational correlation times, which can also be used to characterize complex formation or complex disruption by candidate compound. In such embodiment, since larger complexes of pathogenic protein and its host target have longer rotational correlation times and consequently shorter transverse relaxation times, the NMR signal from the complex decays more rapidly, leading to line broadening. In contrast, if the candidate is effective in preventing complex formation, the pathogenic protein and/or its host target will have shorter rotational correlation times and, thereby, longer transverse relaxation time, which will lead to ¹⁹F line sharpening. By monitoring the peak broadening, the effectiveness of the candidate compound can be assessed.

Using screening techniques such as WaterLOGSY and Saturation Transfer Difference (STD), candidates can be quickly sorted as either binders or non-binders. Further characterization of interactions between ligands and targets can be determined with ¹⁵N—¹H and/or ¹³C—¹H 2D HSQC or other NMR studies which have residue-specific binding resolution. Full structure determination is possible using one or more 2D/3D NMR techniques, such as HCCH-TOCSY, NOESY-HSQC (Nuclear Overhauser Effect (NOE)), HNCA, HNCO, and HN(CA)CO. Finally, in vitro and in vivo studies can be used to determine the effect of ligand candidates on targets by examining the downstream effects.

In one exemplary embodiment, the complex formation of NS1A with CPSF30 (e.g., F2F3) is monitored by directly detecting the chemical shift and relaxation of ¹⁹F isotope of 5-fluoro-tryptophan (5FW) or 4-fluoro-phenylalanine (4FF) labeled NS1A ED, preferably W187, W102 and W203. Since there are only four tryptophan residues in the full-length NS1A protein (one at residue 16 in the N-terminal RBD), the clarity of the data is greatly improved and analysis of the data becomes significantly more efficient. In such embodiment, the chemical shift and relaxation of ¹⁹F isotope can be used to calculate a binding constant between the candidate compound and the NS1A protein. In addition, the ability to measure a binding constant (K_{d}) based upon a titration allows for good comparison between compounds and the natural CPSF30 binding partner. Then, using a pre-mixed sample of 5FW (or 4FF) labeled NS1A ED and CPSF30 fragments (e.g. the F2F3 fragment), a candidate compound can be titrated therein and monitor by NMR whether the candidate compound is breaking the natural NS1A ED/CPSF30 complex. Alternatively, monitor if the candidate compound can out-compete the natural ligand. In particular, the atoms of residues perturbed by binding can be identified and thus the localized interactions between a target pathogen protein (e.g., the NS1A ED) and the candidate compounds can be evaluated.

In another embodiment, the experiments discussed with reference to labeling of NS1A can be performed by placing the ¹⁹F probe (5FW and/or 4FF) on the fragment of CPSF30 (e.g. on the F2F3 fragment). In yet another embodiment, the experiments discussed with reference to labeling of NS1A or CPSF30, can also be performed by placing the ¹⁹F probe (5FW and/or 4FF) on both the NS1A protein and the fragment of CPSF30 (e.g. on the F2F3 fragment).

Although the disclosed methods have been described with reference to a preferred embodiment in designing drugs for treating Influenza A infection, it should be understood by those skilled in the art that the disclosed method can also be applied to other disorders that involved protein-protein interaction. (e.g., disorders involving GLP-1 receptor and binding thereto). The other significant aspect of this invention is due to the fact that current therapies for influenza rely primarily on vaccination and general anti-viral therapies. The current treatment regimen for flu treatment is administration of drugs to reduce symptom severity. In some cases, anti-viral therapies are used but efficacy in most cases appears to be no more than moderate. Drug-resistant variants are seen and the market for influenza therapeutics is small. By designing a drug for a well-conserved region of the NS1A protein, duration and/or severity of the symptoms of influenza A can be significantly reduced.

### Application of the NMR Assay and Peptide Drug Design

The disclosed ¹⁹F NMR assay of a protein complex (e.g. NS1:F2F3 complex) can be used to screen small molecule libraries for inhibitors. In one embodiment, the disclosed ¹⁹F NMR assay can be used for high throughput screening of small molecule libraries to find molecules that either bind to key sites or which disrupt important complexes. The applicability of such assay to high throughput screening is attributed to its ability to examine two or more candidate compounds, preferably between 2 and 100 compounds, at same time (i.e., in parallel) because the assay does not rely on labeling of candidate compounds and can provide high efficiency screening by testing multiple candidate compounds in parallel.

The candidate compound can be successfully designed using the disclosed NMR assay. In one embodiment, as a starting point a candidate compound is selected that has 9 amino acids showing strong alpha helical conformation in silico based on the target site on the NS1 surface described in U.S. 7,709,190 (incorporated by reference in its entirety).

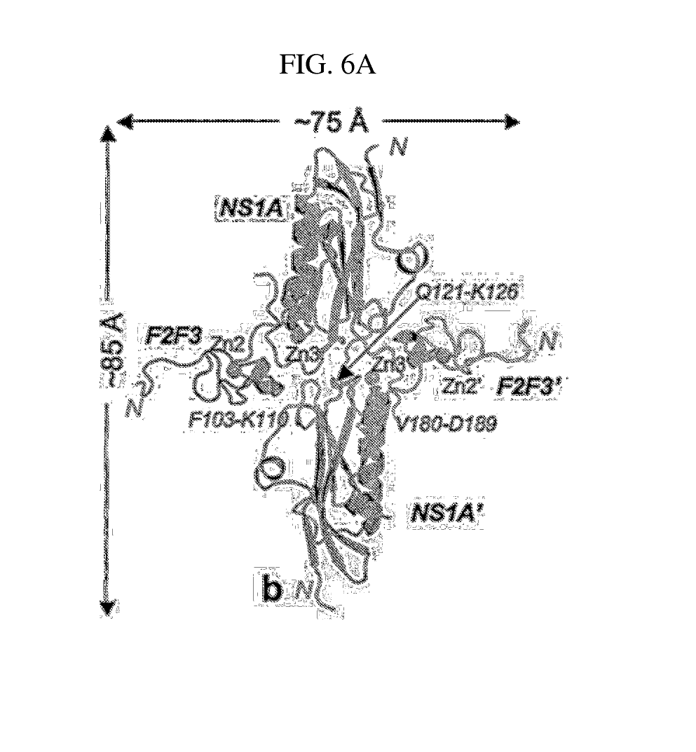

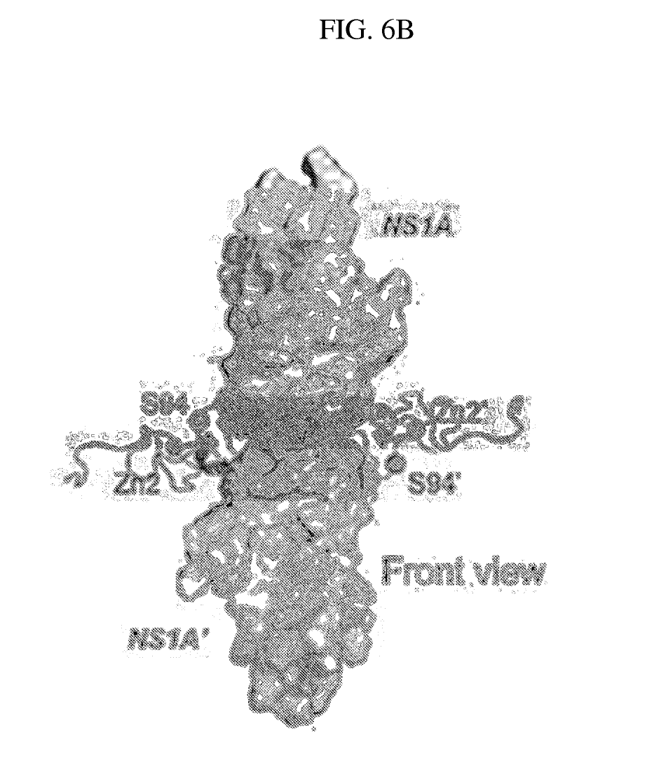

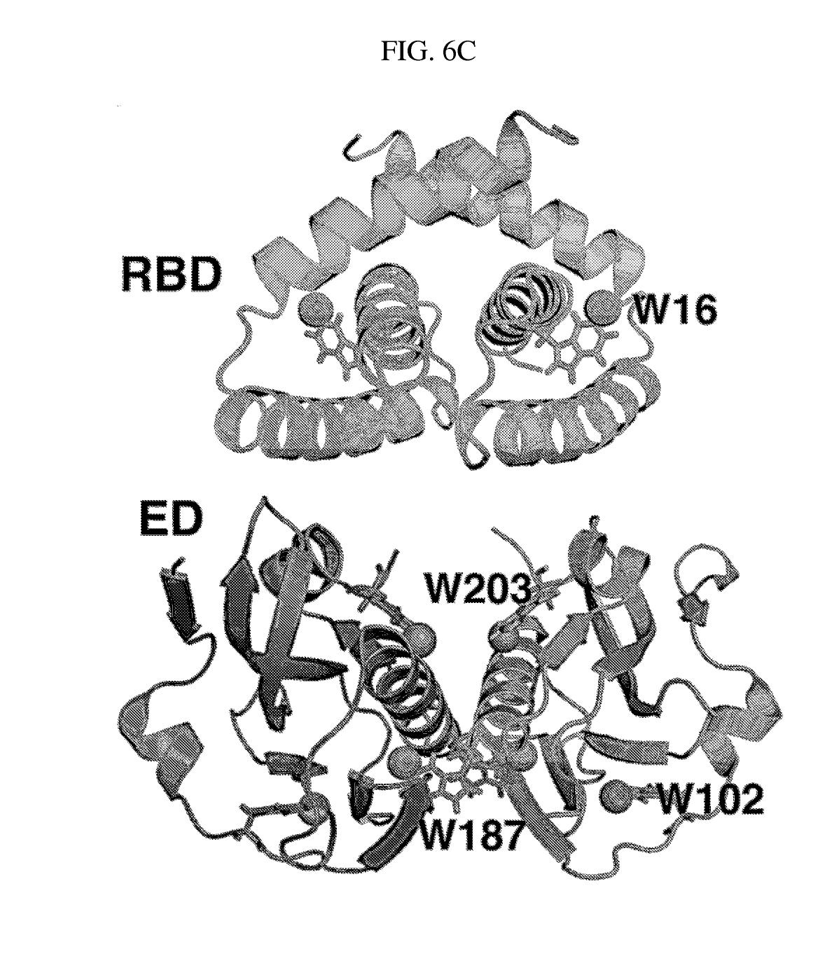

In one exemplary embodiment, the candidate compound is a D-peptide having a sequence dN-Y-F-Y-S-L-F-dQ-G (see FIG. 14). The peptide has the ability to bind NS1A at the site normally used to sequester CPSF30. This same site on NS1A is also responsible for cooperative homodimerization interactions that contribute to an alternative cooperative mechanism by which ED:ED domain interactions contribute to the free energy for binding dsRNA by NS1A. The ¹⁹F tryptophan labeled NS1A ED demonstrates strong chemical shift perturbations of ED residue W187 indicative of binding, whereas W102 and W203 show no chemical shift perturbations upon addition of the designed candidate compound. Addition of F2F3 causes chemical shift perturbations affecting all three tryptophans within the ED (102, 187, 203) because it causes formation of the unique tetameric complex shown in FIG. 6A. Furthermore, the candidate compound binds in the F2F3 binding site which includes W187, but does not have the same extensive set of interactions with NS1A ED as the complete F2F3 molecule, and/or does not induce formation of the large tetrameric complex. In either case—the designed peptide inhibitor binds in the key CPSF-binding site and hence can be an excellent starting point for the development of efficacious inhibitors of the function of the CPSF-binding site/homodimerization site surface epitopes of NS1A ED.

The variants of the above-described D-amino acids-containing candidate can be produced using the same design principles and examined for effectiveness using the disclosed NMR assay. Once the most effective candidate is selected, it can be further tested in vitro and/or in vivo.

### EXAMPLES

### Example 1

Cloning, Expression, Purification, and Sample Preparation: The following three constructs of influenza A/Udorn/307/1972 (H3N2) NS1A (see FIG. 1A) were cloned, expressed, and purified: NS1A(1-215), NS1A(1-73), and NS1A(85-215), hereafter referred to as Ud NS1A, RBD, and ED, respectively following the procedure outlined in FIG. 2. Because the C-terminal 22 residues of full-length Ud NS1A are unstructured and lead to insolubility, these residues were not included in constructs. The three different NS1A constructs were expressed with the following affinity purification tags: i) both the 215-residue NS1A and the 73-residue RBD were fused to an N-terminal 6× His tagged SUMO protein, and ii) the ED was followed by a C-terminal 6× His tag. All single- and double-residue mutants were made using the QuikChange site-directed mutagenesis kit (Stratagene) along with the appropriate primers, and verified by sequencing. The SUMO fusion proteins were cloned into the pSUMO vector (LifeSensors) modified to be ampicillin resistant, whereas the ED constructs were cloned into the pET21_NESG vector. Protein expressions were carried out in Escherichia coli BL21(DE3)-Gold (Agilent) cells containing the rare tRNA codon-enhanced pMgK plasmid and IPTG-inducible T7 polymerase. Cultures were grown in 2 L baffled flasks at 37° C. in MJ9 minimal medium supplemented with ampicillin and kanamycin. For the production of ¹⁵N-labeled proteins ¹⁵NH₄SO₄ (Cambridge Isotope Laboratories) was used as the sole nitrogen source in the MJ9 medium. For 5FW incorporation, cultures were grown until an A₆₀₀ of ≈0.5 units, cooled on ice with addition of 50 mg/L 5-fluoro-DL-tryptophan (Sigma), followed by incubation at 17° C. for 1 h prior to induction with 1 mM IPTG. Protein expression was carried out overnight at 17° C. with shaking at 225 rpm. Cultures were subsequently centrifuged and decanted, and pellets were stored at −80° C. until purification.

All expressed NS1A protein constructs were purified by immobilized metal ion affinity chromatography (IMAC) followed by size-exclusion chromatography in the final buffer for NMR spectroscopy. Pellets were resuspended in 25 mL of nickel affinity column Binding Buffer [50 mM Tris-HCl pH 7.5, 500 mM NaCl, 40 mM imidazole, 1 mM tris(2-carboxyethyl)phosphine, 0.02% NaN₃], followed by sonication, and clarification by centrifugation at 26,000 g for 45 min. After clarification, proteins were purified using an ÄKTAxpress™ system (GE Healthcare) equipped with a 5 mL HisTrap HP affinity column. In the case of NS1A ED and its mutants, samples were immediately loaded onto a HiLoad 26/60 Superdex 75 gel filtration column equilibrated in pH 8 buffer [50 mM Tris-HCl pH 8.0, 30 or 300 mM NaCl, 2.5% (v/v) glycerol, 10 mM DTT]. For the SUMO fusions of NS1A and RBD, an aliquot (1:50-100 mass ratio) of yeast SUMO protease Ulp1 containing an N-terminal 6× His tag expressed and purified in-house was added, and the sample was incubated at 4° C. overnight. Complete SUMO cleavage was confirmed by SDS-PAGE. Cleaved NS1A was then purified using a HiLoad 26/60 Superdex 200 gel filtration column equilibrated in pH 8 buffer. Due to its comparable size and resulting similar retention time, free NS1A RBD could not be separated from the cleaved SUMO tag using size-exclusion chromatography. Consequently, the sample was buffer exchanged with a HiPrep 26/10 desalting column into Rebinding Buffer [50 mM Tris-HCl pH 7.5, 500 mM NaCl, 1 mM tris(2-carboxyethyl)phosphine, 0.02% NaN₃], and passed over a 5 mL HisTrap HP affinity column to remove cleaved SUMO tag and SUMO protease. The flow-through containing RBD was finally purified using a HiLoad 26/60 Superdex 75 gel filtration column equilibrated in pH 8 buffer. The purities of all NS1A proteins used in this study were confirmed by SDS-PAGE and MALDI-TOF mass spectrometry. Unless otherwise indicated, samples of 5FW-labeled NS1A constructs for NMR and analytical ultracentrifugation were prepared in low (30 mM NaCl) or high (300 mM NaCl) salt pH 8 buffer containing 10% (v/v) ²H₂O and concentrated by ultrafiltration (Amicon, Millipore). The buffer conditions were carefully optimized in order to promote dimerization of the isolated ED (low salt) or enhance the solubility of full-length NS1A (high salt). Samples for deuterium isotope effect NMR experiments were prepared by performing three rounds of 1:10 dilution with pH 8 buffer in 90% ²H₂O followed by concentration by ultrafiltration, resulting in a final ²H₂O concentration of ≈90% (v/v).

Efficiency of 5FW Incorporation: To assess the efficiency of 5FW incorporation, aliquots of NS1A ED expressed in various labeling media were diluted in 100 mM Tris-HCl pH 8.0, 10 mM CaCl₂, 8 M urea, 10 mM DTT and denatured at 60° C. for 25 min. Proteolysis was carried out by adding 0.1 μg chymotrypsin (Sigma), and incubating the mixture at 37° C. for 2 h. Proteolytic digestion was arrested by addition of 2 μL 5% (v/v) trifluoroacetic acid (TFA), and complete digestion was confirmed by SDS-PAGE. The resulting peptide digest was mixed (1:100) with 10 mg/mL α-Cyano-4-hydroxycinnamic acid (CHCA) in 50% acetonitrile, 1% TFA, and 1 μL was plated for reflected MALDI TOF/TOF on an ABI-MDS SCIEX 4800 mass spectrometer. Mass spectrometry spectral data were analyzed using Data Explorer software (Applied Biosystems). Fluorinated peptides were selected using the m/z protease digest prediction tool Protein Prospector and confirmed with tandem (MS/MS) mass spectrometry. High levels (≧90%) of biosynthetic 5FW incorporation were consistently observed.

### Example 2

One-dimensional (1D) ¹⁹F NMR spectroscopy was performed locked and at 20° C. on a Varian INOVA 500 MHz spectrometer equipped with a room temperature 5-mm ¹H/¹⁹F switchable probe at a frequency of 470.18 MHz. All ¹⁹F NMR spectra were acquired using VNMRJ 2.1B and referenced to external neat trichlorofluoromethane, CFCl₃. Typical 1D ¹⁹F NMR acquisition parameters were as follows: a 20,000 Hz sweep width (42.5 ppm), a 0.35 s acquisition time, a 5 s relaxation delay time, and a 5.0 μs 90° pulse length. 1D ¹⁹F NMR spectra were processed with 20 Hz exponential line broadening and displayed using matNMR. Two-dimensional (2D) ¹H—¹⁵N TROSY-HSQC spectra were acquired locked and at 25° C. on a Bruker AVANCE 800 MHz NMR spectrometer equipped with a 5-mm TXI cryoprobe, referenced to internal 2,2-dimethyl-2-silapentane-5-sulfonic acid (DSS), processed with NMRPipe, and displayed using Sparky.

¹⁹F NMR of biosynthetically incorporated fluorinated amino acids and analogs were used to monitor protein self-association. As illustrated in FIGS. 1B and 6C, the NS1 protein from influenza A has four tryptophan residues in the entire protein sequence, one in the N-terminal dsRNA binding domain (RBD) and three in the C-terminal effector domain (ED).

Previous biophysical studies established the importance of Trp-187 for ED dimerization in solution via an intermolecular helix-helix interaction (Aramini et al., 2011, J. Biol Chem 286, 26050-26060; incorporated herein by reference in its entirety). Incorporation of 5FW in NS1A ED combined with ¹⁹F NMR affords a sensitive probe of this dimer interface, thereby providing unique structural and biophysical insights into this biologically important binding surface.

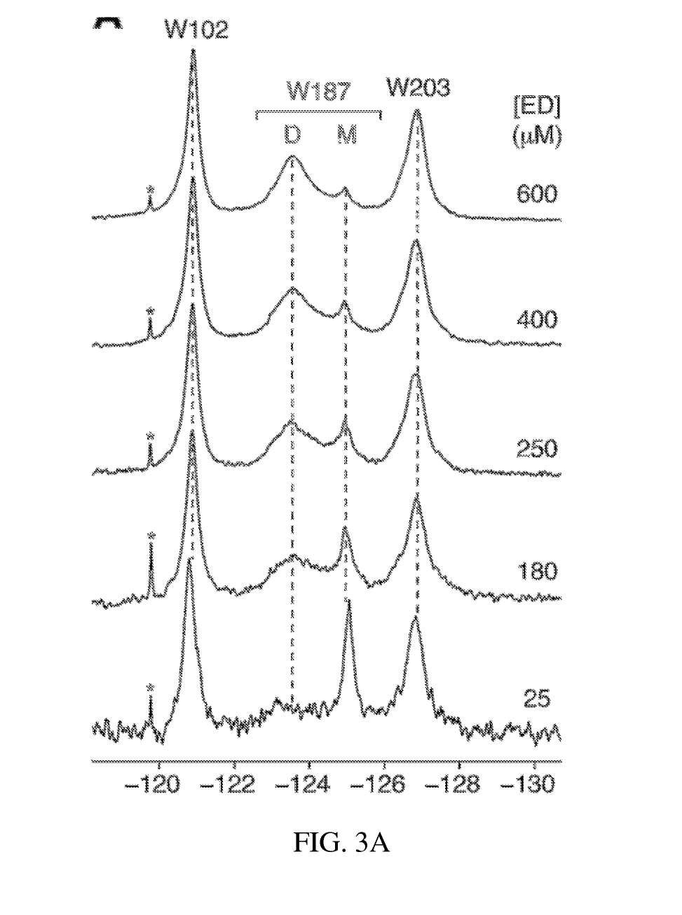

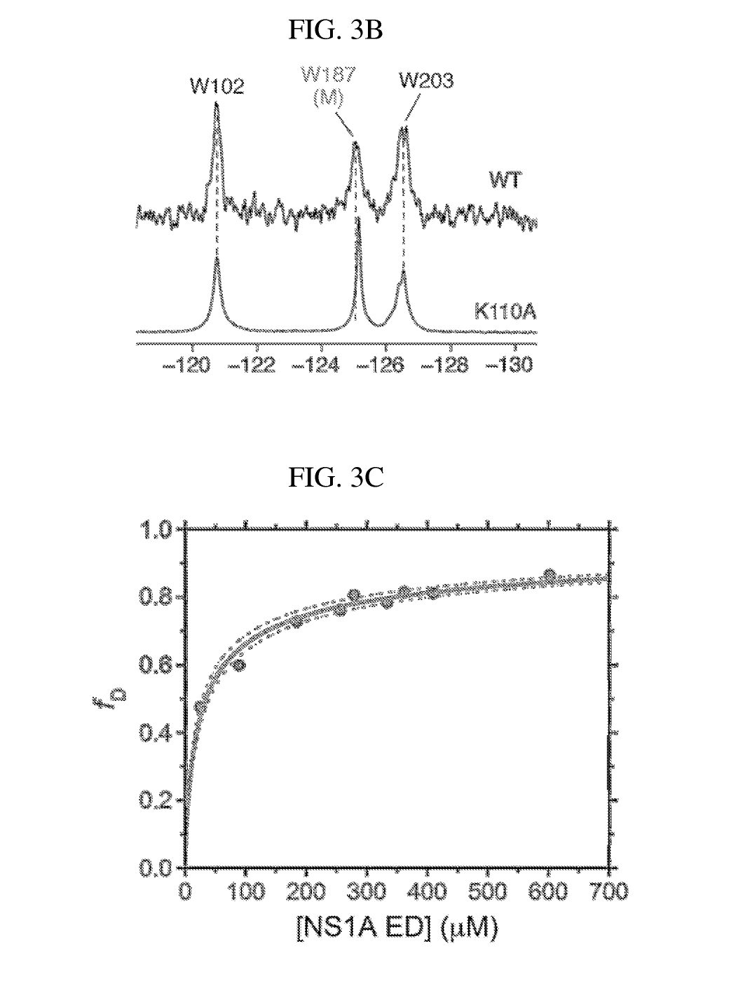

To obtain the dissociation constant for homodimerization, K_{d}, of NS1A ED by ¹⁹F NMR, values of the fraction of dimer, f_{D}, at each ED concentration were obtained from the volumes of Trp 187 dimer and monomer ¹⁹F signals shown in FIG. 3A determined by Lorentzian line fitting using MATLAB 7.12.0 (MathWorks). Assuming a two-state dimer-to-monomer equilibrium, the K_{d} is related to f_{D}, as well as the fraction of monomer, f_{M}, and total protein concentration, P_{T}, according to Eq. 1:

K_{d}=2P_{T}f_{M}²/fD (1)

Expressing this relationship in terms of f_{D} as a function of P_{T} and K_{d} yields a quadratic expression Eq. 3, which we employed to fit the concentration dependence of f_{D} to compute K_{d} by non-linear least squares fitting using MATLAB 7.12.0 as shown in FIG. 3C.

f_{D}=[4P_{T}+K_{d}−\sqrt{K_{d}²+8P_{T}K_{d}}]4P_{T} (2)

The wild type and 5FW labeled NS1A ED exhibited comparable K_{d} values in low salt pH 8 buffer (K_{d}=12±6 μM and 7±5 μM for wild type and 5fW NS1A ED, respectively). As shown in this example, the incorporation of 5FW-labeled NS1A ED results in only minor biophysical perturbations, allowing to directly monitor the dimer⇄monomer equilibrium of this domain and determine a dissociation constant, K_{d}, for this equilibrium.

Furthermore, the solvent exposure of tryptophan side chains in the protein, and the presence of conformational exchange dynamics at the interface were evaluated in the 5FW-labeled NS1A ED (Aramini et al., 2014, Structure, in the press). In particular, dilution of dimeric 5FW-labeled NS1A ED resulted in a progressive decrease in the broad resonance for Trp187 and concomitant increase in a second sharper upfield resonance (see FIG. 3A). This upfield-shifted resonance for Trp187 is also observed for the K110A mutant of Ud NS1A ED, a mutant of this protein domain that is monomeric (see FIG. 3B). Hence, this sharp upfield-shifted resonance corresponds to Trp187 in the exposed monomeric state.

### Example 3

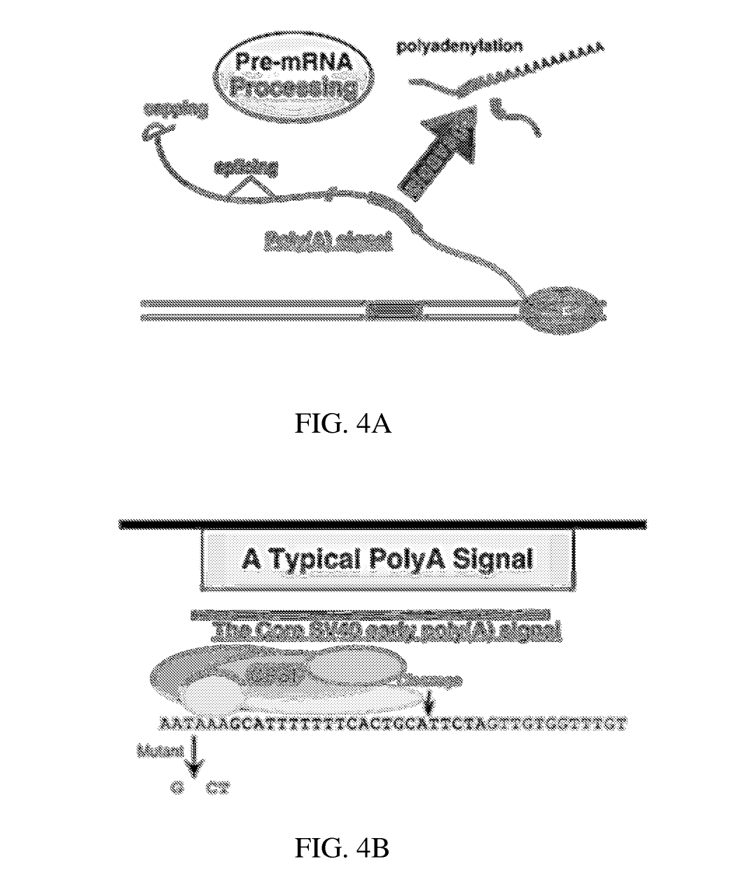

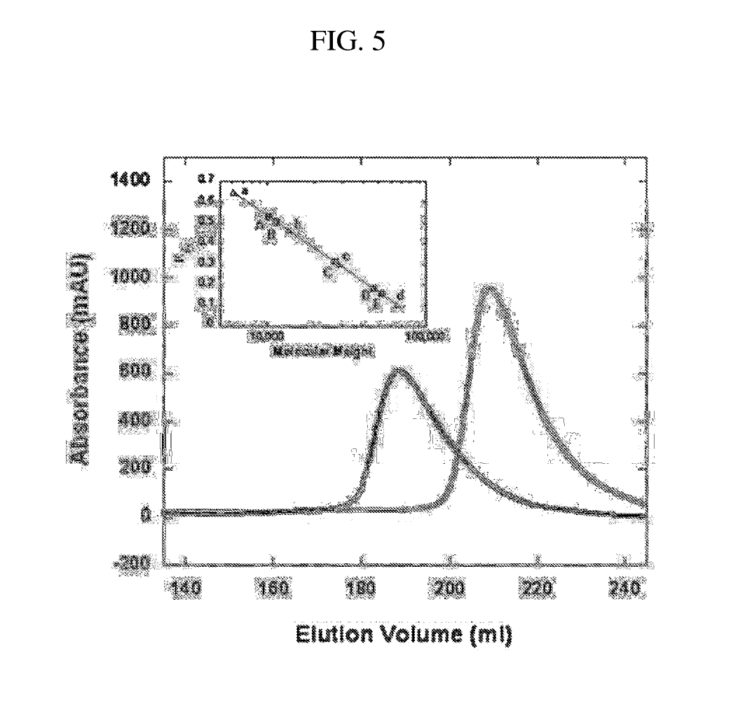

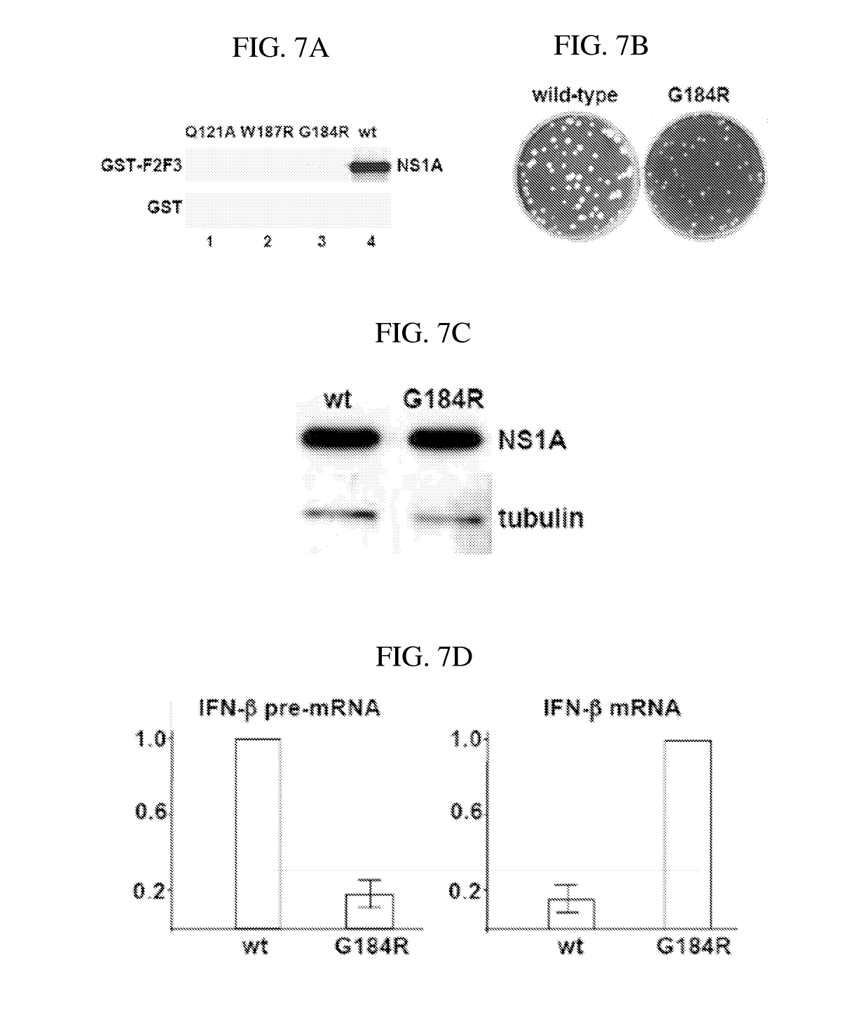

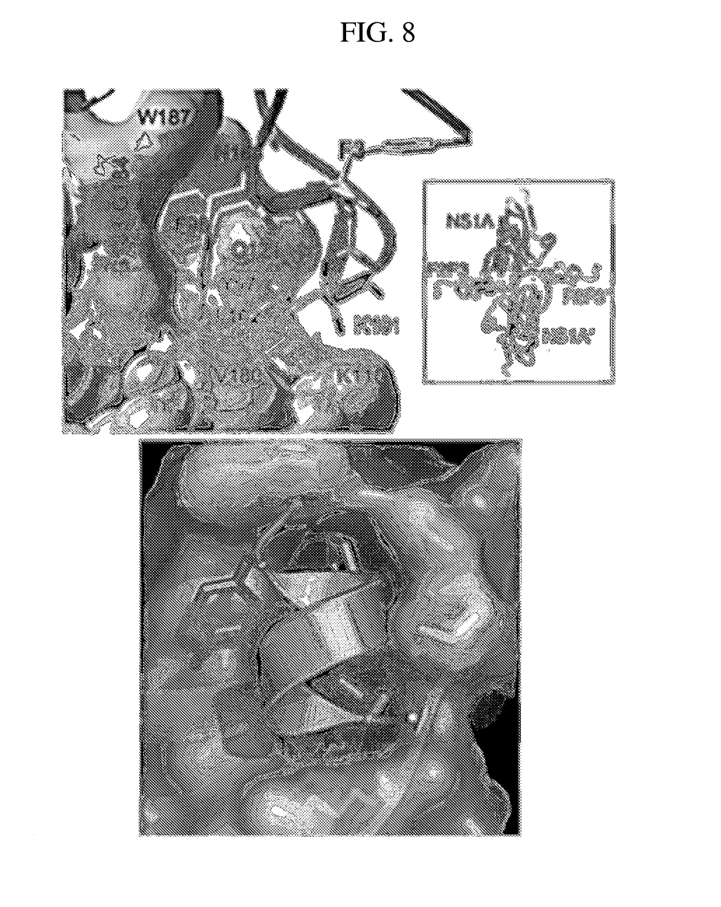

¹⁹F NMR was used to monitor formation of a protein-protein complex between NS1A ED and CPSF30. The combination of selective fluorinated amino acid incorporation and ¹⁹F NMR spectroscopy is ideally suited for investigations of protein-protein complex formation. During influenza A viral infection, NS1A targets host CPSF subunit 30 (CPSF30), a 30-kDa subunit of the cellular Cleavage and Polyadenylation complex, thereby suppressing the 3′ processing of pre-mRNAs coding for antiviral response proteins, including beta-Interferon as shown in FIGS. 4 and 7. A small tandem zinc-finger domain from CPSF30 (F2F3) is the minimal unit required for interaction with NS1A ED, forming a heterotetrameric complex with this NS1A domain (see FIGS. 5 and 8). The crystal structure of the complex described in Das, K., et al. (Proc. Natl. Acad. Sci. 105, 2008, 13093-13098; incorporated herein by reference in its entirety) is shown in FIGS. 6A and 6B. The in vivo mutagenesis experiments also confirmed that mutation of residues in the ED:F2F3 interface that abolish complex formation both severely attenuate viral growth and render the mutated virus unable to suppress the processing of host cell interferon mRNA (Das, 2008) (see FIG. 7).

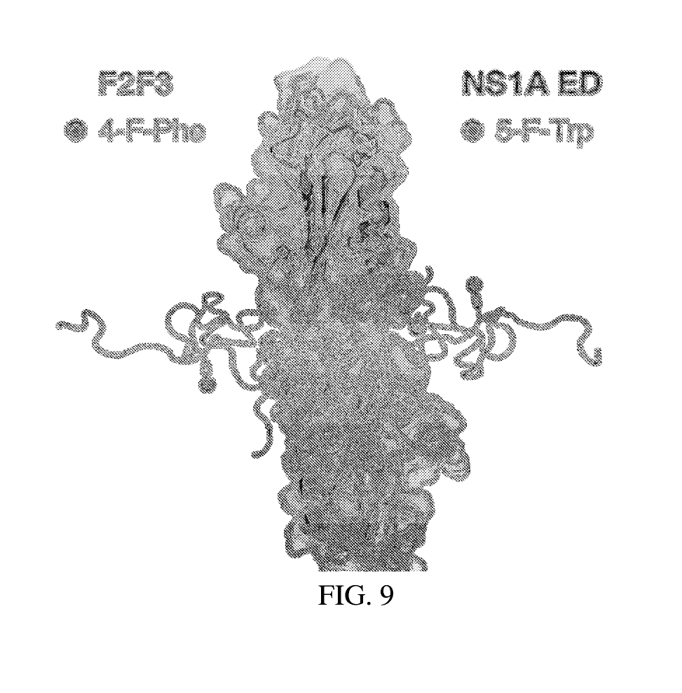

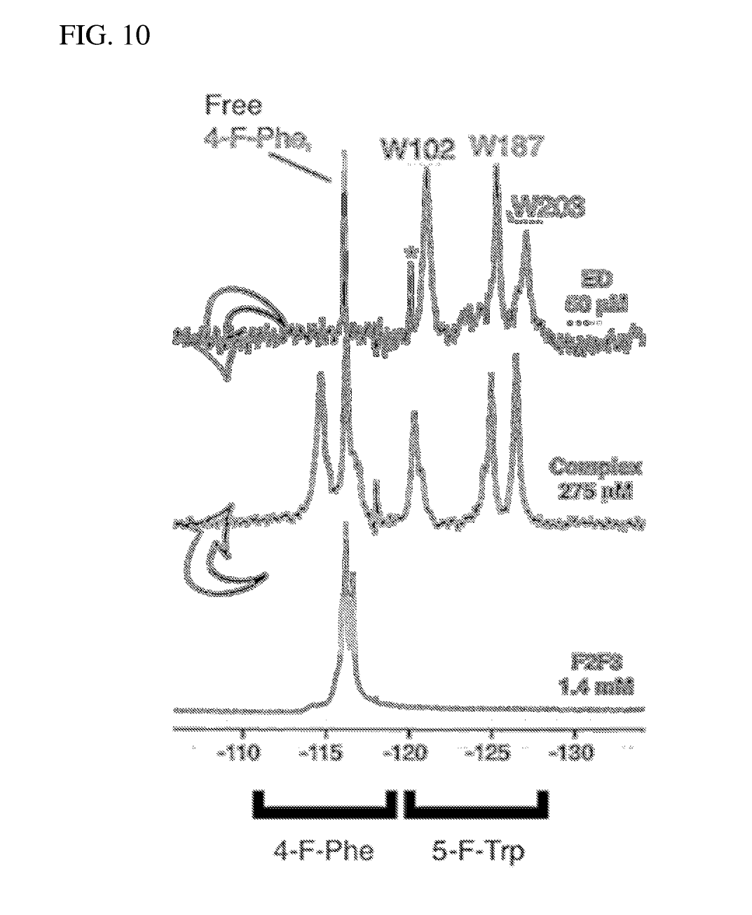

Using fluorinated amino acid incorporation plus ¹⁹F NMR, fluorinated ED:F2F3 complexes were prepared and examined (see FIG. 9). As shown in FIG. 10, by exploiting the broad chemical shift range for ¹⁹F, it was possible to combine 5FW-labeled NS1A ED with 4FF-labeled F2F3 to generate a double fluorinated NS1A ED:F2F3 complex featuring ¹⁹F signals for 5FW and 4FF residues within the same ¹⁹F NMR spectrum. The ¹⁹F chemical shift changes upon complex formation provide an assay for the complex which can be used for screening molecules that inhibit this interaction.

### Example 4

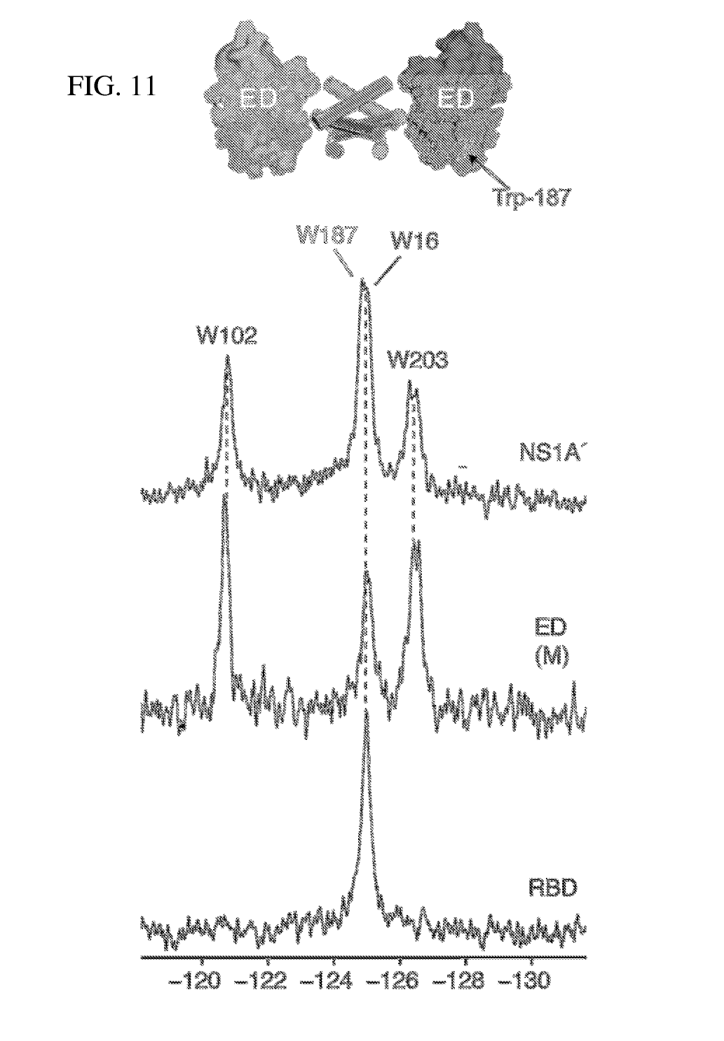

In this example, ¹⁹F NMR of fluorinated amino acids was used to monitor protein self-association: lack of intermolecular ED:ED interactions within full length NS1A. The combination of fluorination and ¹⁹F NMR was extended to 5FW-labeled full-length NS1A. The ¹⁹F NMR spectrum of full-length NS1A protein featured four ¹⁹F Trp resonances, which can be assigned by comparison with the ¹⁹F spectra of its isolated dimeric RBD and monomeric ED domains (see FIG. 11). At subaggregate NS1A concentrations (up to 50 μM) the ¹⁹F resonance corresponding to Trp187 is fully solvent exposed, meaning that Trp187 is not involved in either intra- or interdimeric ED:ED interactions (Aramini et al., 2014, Structure, in the press). Moreover, this fluorinated full-length construct can be applied to ¹⁹F investigations of dsRNA binding by the full-length protein and reagents that interfere with this interaction.

### Example 5

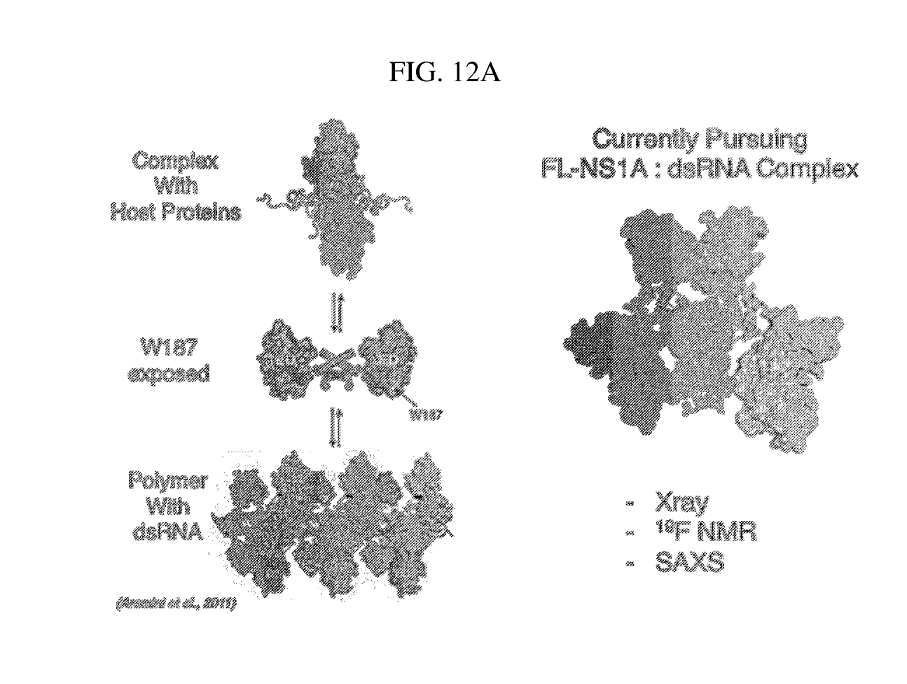

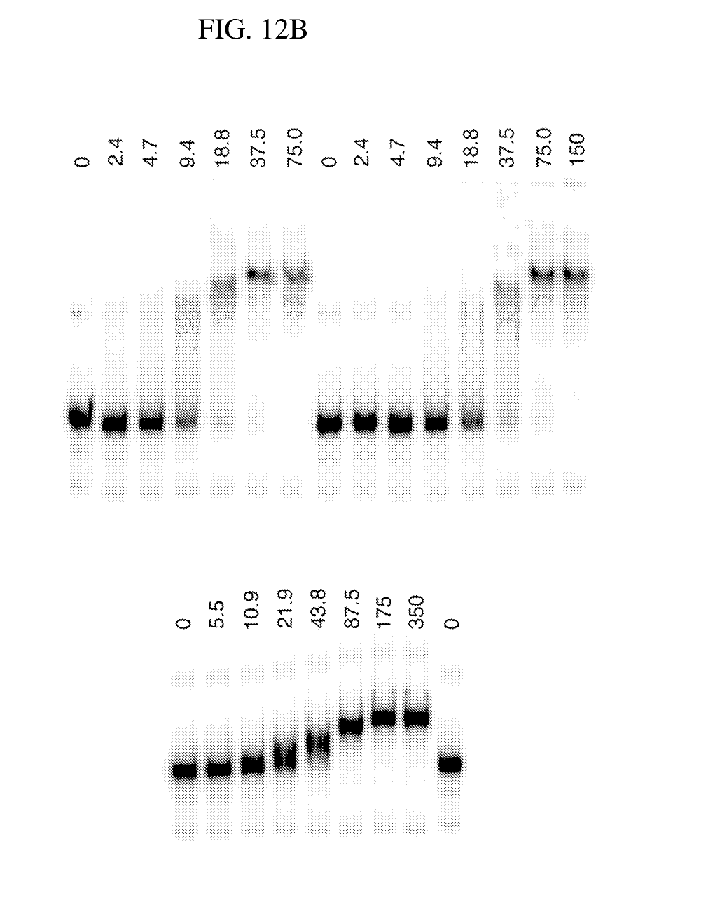

Homodimerization of NS1A ED is a driving force for cooperative dsRNA binding: another important target for antiviral drug discovery. Intermolecular ED:ED interactions in NS1A play a critical role in the established cooperative, high affinity binding of dsRNA by NS1A. This function is required for the virus to sequester transient dsRNA formed during viral replication, which would otherwise trigger a strong antiviral response via the 2′-5′ oligonucleotide A synthetase/RNase L pathway. Hence, suppressing or blocking the interaction between dsRNA and the RBD of NS1A, which features a highly conserved dsRNA-binding epitope across all influenza A and B viruses, provides another target for anti-influenza drug development. Electrophoretic gel mobility assays (EMSA) shown in FIG. 12B revealed that Trp187 in NS1A, and consequently intermolecular ED:ED interactions, is required for cooperative dsRNA binding by the full-length protein (see FIG. 12A). Mutating this residue reduced this cooperativity and removing the ED altogether further reduced both dsRNA binding affinity and cooperativity. Hence, our model for the multi-functional activity of influenza NS1A features a spatially and temporally regulated equilibrium between an exposed Trp-187 surface that becomes buried upon interaction with i. host target proteins, such as CPSF30, and ii. dsRNA in an oligomeric fashion (see FIG. 12A). Formation of this complex can be directly assayed using ¹⁹F NMR.

### Example 6

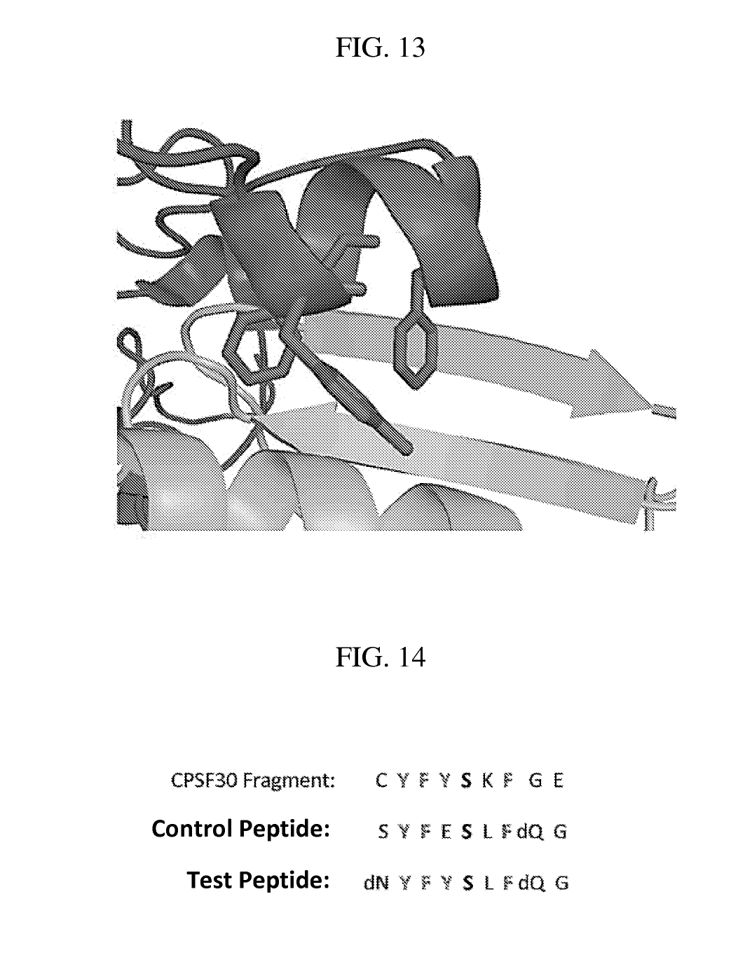

The disclosed ¹⁹F NMR assay was used to detect site-specific protein interactions with peptides or small molecules. It was known that the NS1A protein binds to an alpha helix of the CPSF30 protein with the sequence CYFYSKFGE (see FIG. 13). Thus, using this helix as template, a peptide dNYFYSLFdQG (Test Peptide; SEQ ID NO. 1) was designed and tested against a control sequence (see FIG. 14). The peptides were synthesized at the Tufts University Peptide Core Facility with roughly 20 mg pure peptide received per design. 10 ml of 100 uM peptide solution was made in phosphate buffered saline.

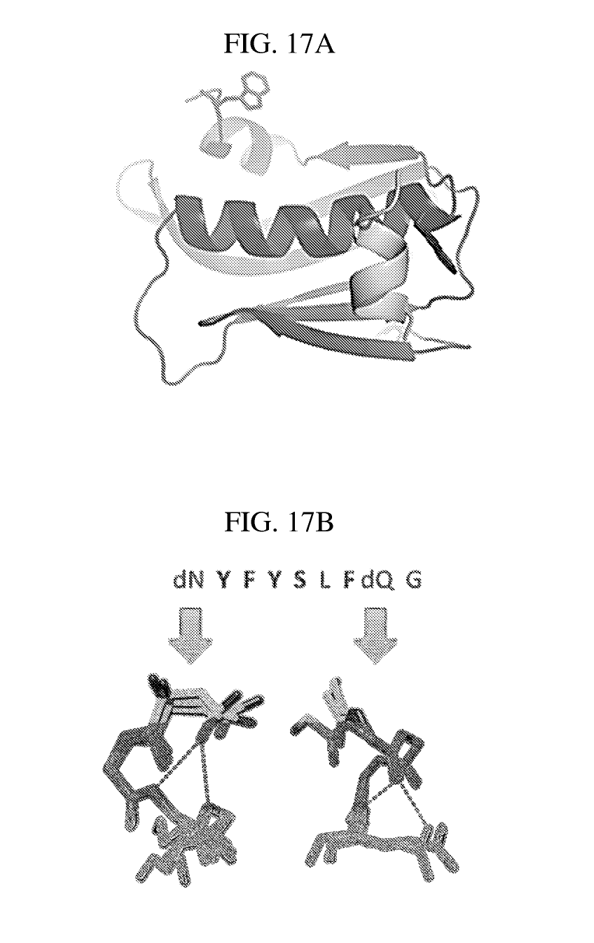

Based on the similarity to the CPSF30 fragment (see FIG. 13), the peptide with sequence of dAsn-Tyr-Phe-Tyr-Ser-Leu-Phe-dGln-Gly was predicted to bind the CPSF30-F2F3 binding site of the NS1A protein (also see FIG. 17A) This same site on NS1A ED contributes to the cooperatively of dsRNA-binding by full length NS1A. Hence the inhibitor peptide has the potential to block the innate immune response suppression activity of NS1A based on two distinct functions of this surface site of the NS1 ED.

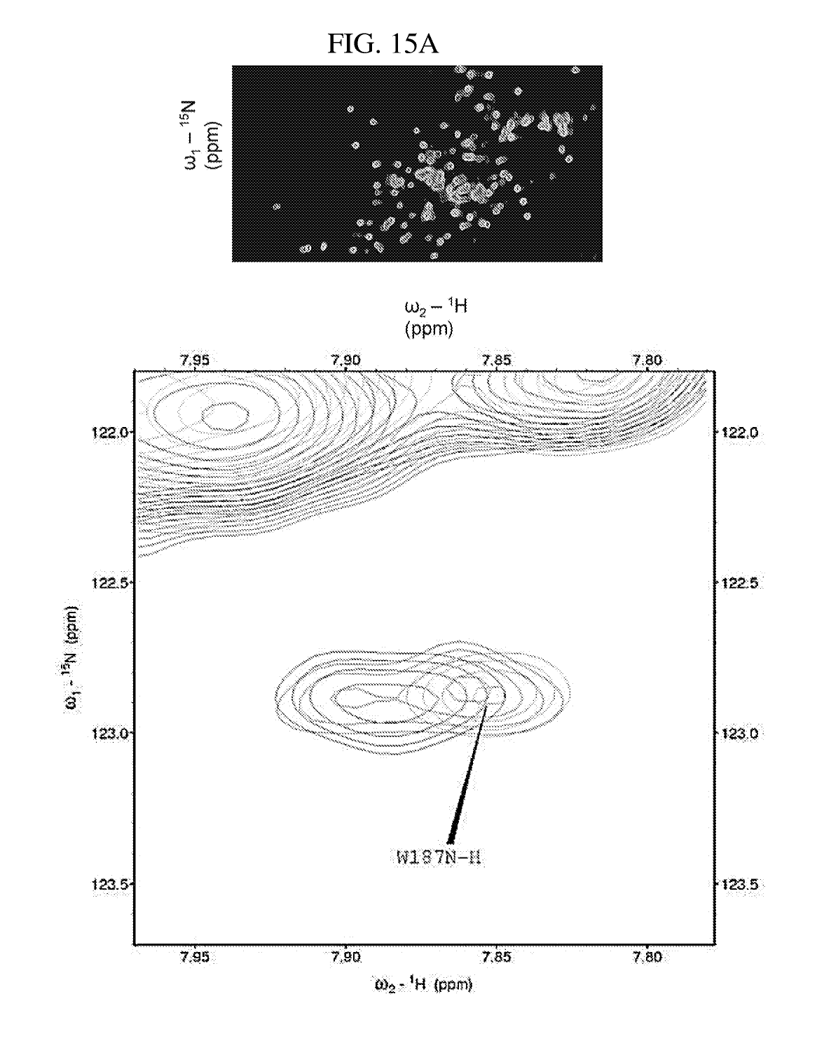

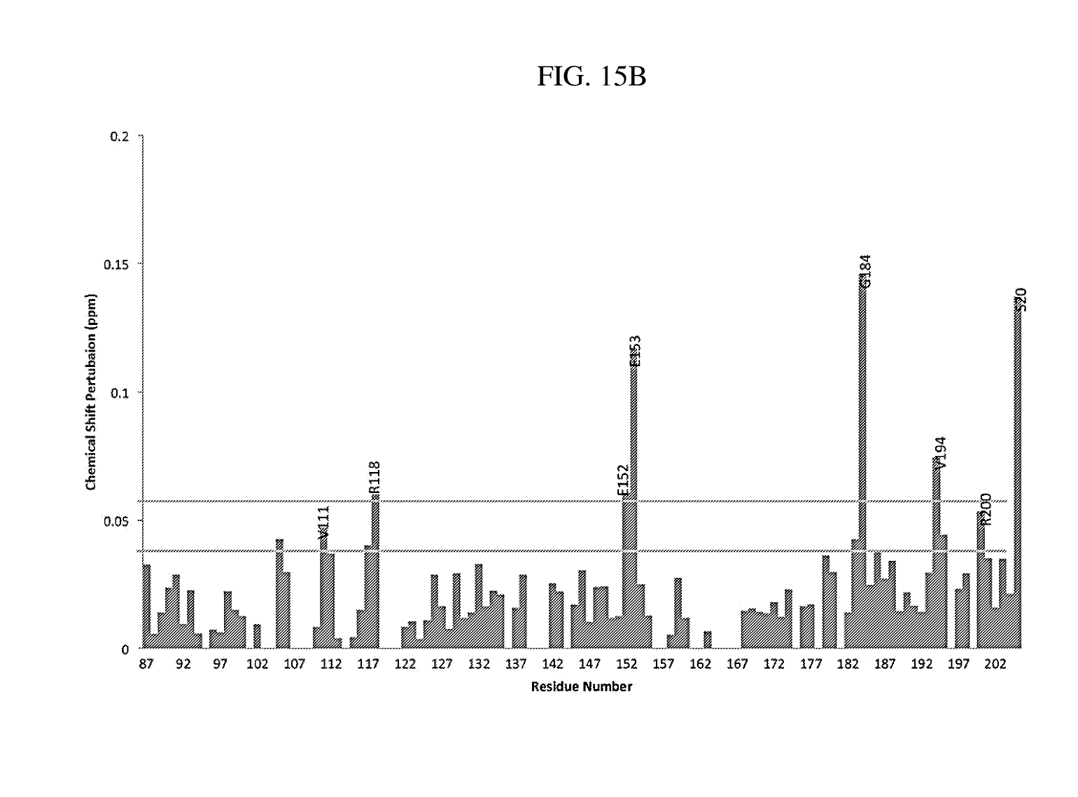

2D ¹⁵N—¹H HSQC spectrum was recorded using both peptides and ¹⁵N-enriched NS1A ED to determine binding localization of the peptides to the ED (see FIG. 15A). Sequence-specific ¹⁵N and ¹H resonance assignments for NS1 ED have been determined as shown in Example 1. The peptide showed slow-exchange, indicative of strong binding, to the binding site as well as fast-exchange, or weak binding to other residues of the ED. A chemical shift perturbation chart shown in FIG. 15B was generated using the measured chemical shifts seen in FIG. 15A. A titration of test and control peptides to NS1A ED was performed. Using 200 uM ED and 0, 50, 100, 200, and 300 uM concentrations of the test peptide, slow-exchange vs. fast-exchange residues were noted. The residue W187 was shown to feature slow-exchange indicative of tight binding.

Using D-amino acids to cap the N and C-terminal ends allowed a small peptide to adopt the proper secondary structure conformation to enable binding as shown in FIG. 17B (Rodriguez-Granillo, A., et al 2011, J. Am. Chem. Soc. 133, 18750-9). Benefits of using a designed peptide include good pharmacokinetic and pharmacodynamics properties, low immunogenicity, and low toxicity. (see FIGS. 17 and 18)

¹⁹F NMR is an ideal assay for characterizing binding and inhibition of the NS1A protein. Using 1D ¹⁹F NMR, it was possible to identify the binding location of a designed peptide according to its proximity to the nearest ¹⁹F labeled Trp amino acids (see FIG. 16A vs. FIG. 16B). Using this data, it was also possible to quantify binding affinity. The designed peptide dAsn-Tyr-Phe-Tyr-Ser-Leu-Phe-dGln-Gly was shown to bind near the Trp187 amino acid residue in the CPSF30 binding site of NS1 A protein, since it exhibits a strong ¹⁹F chemical shift change for the Trp187 resonance, and demonstrates no significant impact on the Trp102 and Trp203 resonances.

It will be appreciated by persons skilled in the art that the present invention is not limited to what has been particularly shown and described herein above. Rather, the scope of the present invention is defined by the claims that follow. It should further be understood that the above description is only representative of illustrative examples of embodiments. For the reader's convenience, the above description has focused on a representative sample of possible embodiments, a sample that teaches the principles of the present invention. Other embodiments may result from a different combination of portions of different embodiments.

1. A method of identifying an inhibitor of protein-target interaction in vitro comprising:
providing a reaction system comprising
(i) at least a protein or a target-binding portion thereof,
(ii) at least a target or a protein-binding portion thereof, and
(iii) a candidate compound,
wherein either or both protein and target or portions thereof are labeled with ¹⁹F isotope; and
detecting binding between said protein and said target by monitoring changes in the nuclear magnetic resonance attributed to the ¹⁹F isotope;
wherein reduced binding in the presence of the candidate compound relative to a control is indicative of activity of the compound in inhibiting the protein-target interaction.

2. The method of claim 1, wherein the target is selected from a group consisting of protein, nucleic acid, and small molecule.

3. The method of claim 2, wherein the protein is a pathogenic protein.

4. The method of claim 3, wherein the pathogenic protein is NS1.

5. The method of claim 4, wherein the pathogen is influenza A virus.

6. The method of claim 4, wherein the pathogenic protein is NS1 and the target is NS1 and the protein-target interaction is homodimerization of NS1 effector domain.

7. The method of claim 4, wherein the target is CPSF30.

8. The method of claim 7, wherein the target is a F2F3 fragment of CPSF30.

9. The method of claim 1, wherein either or both protein and target or portions thereof are labeled with 5-fluoro-tryptophan (5FW) and/or 4-fluoro-phenylalanine (4FF).

10. A method of identifying an inhibitor of influenza A virus comprising:
providing a reaction system comprising
(i) at least NS1A protein or a CPSF30-binding portion thereof,
(ii) at least CPSF30 or an NS1A-binding portion thereof, and
(iii) a candidate compound,
wherein either the CPSF30-binding portion of NS1A, the NS1A-binding portion of CPSF30, or the CPSF30-binding portion of NS1A and the NS1A-binding portion of CPSF30 are labeled with 5-fluoro-tryptophan (5FW) and/or 4-fluoro-phenylalanine (4FF); and
detecting binding between the at least a CPSF30-binding portion of NS1A and the at least a NS1A-binding portion of human CPSF30, wherein reduced binding in the presence of the candidate compound relative to a control is indicative of activity of the compound against influenza A virus.

11. The method of claims 1 and 10, wherein the control comprising
(i) at least a protein or a target-binding portion thereof, and
(ii) at least a target or a protein-binding portion thereof,
wherein either or both protein and target or portions thereof are labeled with ¹⁹F isotope.

12. The method of claim 11, wherein the detecting step is carried out by ¹⁹F NMR.

13. The method of claim 12, wherein the detecting step is carried out by detecting chemical shift perturbation.

14. The method of claim 13, further comprising varying the amount of the candidate compound in the reaction system.

15. The method of claim 14, wherein the candidate compound is a peptide.

16. The method of claim 15, wherein the peptide comprises at least one D-amino acid.

17. The method of claim 16, wherein the candidate compound is a peptide containing an alpha helical conformation.

18. The method of claim 17, wherein the peptide is 9-50 amino acids in length.

19. The method of claim 18, wherein the peptide is 9-20 amino acids in length.

20. The method of claim 19, wherein the peptide is 9 amino acids in length.

21. A method of preventing or treating influenza A infection comprising:
identifying a patient in need of such prevention or treatment, and
administering to said patient a first therapeutic agent comprising a therapeutically effective amount of a candidate compound identified in claim 1 or the pharmaceutical composition thereof.

22. The method of claim 6, wherein Trp187 residue is mutated to attenuate oligomerization of full-length NS1A.


SEQUENCE LISTING (10 sequences)

SEQ ID NO: 1 - protein, 9 aa, Artificial
  Synthetic peptide
XYFYSLFXG

SEQ ID NO: 2 - protein, 269 aa, Homo Sapiens
MQEIIASVDHIKFDLEIAVEQQLGAQPLPFPGMDKSGAAVCEFFLKAACGKGGMCPFRHISGEKTVVCKHWLRGLCKKGDQCEFLHEYDMTKMPECYFYSKFGECSNKECPFLHIDPESKIKDCPWYDRGFCKHGPLCRHRHTRRVICVNYLVGFCPEGPSCKFMHPRFELPMGTTEQPPLPQQTQPPAKQSNNPPLQRSSSLIQLTSQNSSPNQQRTPQVIGVMQSQNSSAGNRGPRPLEQVTCYKCGEKGHYANRCTKGHLAFLSGQ

SEQ ID NO: 3 - protein, 237 aa, Influenza A virus
MDSNTVSSFQVDCFLWHVRKQVVDQELGDAPFLDRLRRQDKSLRGRGSTLGLNIEAATHVGKQIVEKILKEESDEALKMTMASTPASRYITDMTIEELSRDWFMLMPKQKVEGPLCIRIDQAIMDKNIMLKANFSVIFDRLETLILLRAFTEEGAIVGEISPLPSFPGHTIEDVKNAIGVLIGGLEWNDNTVRVSKTLQRFAWGSSNENGRPPLTPKQKRKMARTARSKVRRDKMAD

SEQ ID NO: 4 - protein, 230 aa, Influenza A
MDPNTVSSFQVDCFLWHVRKRVADQELGDAPFLDRLRRQDKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 5 - protein, 230 aa, Influenza A
MDSNTITSFQVDCYLWHIRKLLSMRDMCDAPFDDRLRRQDKALKGRGSTLGLDLRVATMEGKKIVEDILKSETDENLKIAIASSPAPRYITDMSIEEISREWYMLMPRQKITGGLMVKMDQAIMDKRITLKANFSVLFDQLETLVSLRAFTDDGAIVAEISPIPSMPGHSTEDVKNAIGILIGGLEWNDNSIRASENIQRFAWGIRDENGGPPLPPKQKRYMARRVESEV

SEQ ID NO: 6 - protein, 215 aa, Influenza A
MDSNTVSSFQVDCFLWHVRKRFADQELGDAPFLDRLRRQDKSLRGRGNTLGLDIETATRAGKQIVERILEGESDKALKMPASRYLTDMTLEEMSRDWFMLMPKQKVAGSLCIKMDQAIMDKTIILKANFSVIFDRLETLILLRAFTEEGAIVGEISPLPSLPGHTGEDVKNAIGVLIGGLEWNDNTVRVTETIQRFAWRNSDEDGRLPLPPNQKR

SEQ ID NO: 7 - DNA, 40 bp, Artificial
  Synthetic
aataaagcatttttttcactgcattctagttgtggtttgt

SEQ ID NO: 8 - protein, 9 aa, Artificial
  Synthetic peptide
CYFYSKFGE

SEQ ID NO: 9 - protein, 8 aa, Artificial
  Synthetic peptide
SYFESFXG

SEQ ID NO: 10 - protein, 215 aa, Influenza A virus
MDSNTVSSFQVDCFLWHVRKQVVDQELGDAPFLDRLRRQDKSLRGRGSTLGLNIEAATHVGKQIVEKILKEESDEALKMTMASTPASRYITDMTIEELSRDWFMLMPKQKVEGPLCIRIDQAIMDKNIMLKANFSVIFDRLETLILLRAFTEEGAIVGEISPLPSFPGHTIEDVKNAIGVLIGGLEWNDNTVRVSKTLQRFAWGSSNENGRPPLT